(19) United States
(12) Patent Application Publication
(10) Pub. No.: US 2002/0124843 A1
(43) Pub. Date: Sep. 12, 2002

(54) Eye medication delivery system

(76) Inventors: Jeffry B. Skiba, Oracle, AZ (US); Steven T. Dodd, Coppell, TX (US)

Correspondence Address: Gardere Wynne Sewell LLP, 3000 Thanksgiving Tower, Suite 3000, 1601 Elm Street, Dallas, TX, 75201-4767, US

(21) Appl. No.: 10/060,754

(22) Filed: Jan. 30, 2002

Related U.S. Application Data
(60) Provisional application No. 60/265,744, filed on Feb. 1, 2001.

(51) Int. Cl. A61M 11/00

(52) U.S. Cl. 128/200.18

(57) ABSTRACT

An apparatus for delivering medicine to one or more eyes is provided. The apparatus includes a mask worn around the eyes. The mask has a substantially transparent mask face and one or more fog outlets proximate to the eyes. An atomizer nebulizes medicine into a medicine-carrying fog such that the fog discharges from the fog outlets to deliver medicine carried by the fog to one or more eyes. The momentum of the liquid fog droplets is low so that the eyes do not adversely react to the droplet impact, yet the droplet density is sufficient to deliver an effective amount of medication. Additionally, the apparatus may include a power supply, a propellant source and one or more conduits. Methods of delivering medication to the eyes are also provided.

This application is a claims priority from co-pending Provisional Patent Application Serial No. 60/265,744, filed Feb. 1, 2001.

### FIELD OF THE INVENTION

The present invention relates to ophthalmology and, in particular, to an apparatus and methods for administering medication to the eyes.

### BACKGROUND OF THE INVENTION

Without limiting the scope of the invention, its background is described in connection with eye treatment, as an example, although the present invention contemplates the treatment of any biological tissue either human or veterinary.

Most people who require medication into a sensitive area such as an eye, a burn or a wound, are reluctant to have liquids, creams, or gels placed directly on the tissue. In particular, inspecting and treating the human eye is an usually difficult task because the eye is a very sensitive area of the body. It is often difficult for a physician, ophthalmologist or oculist to treat and inspect the eye because the patient is unable to comfortably maintain his or her eyelids in an open, unblinking, position. Usually the eye tears up and the eye may retract when the eyelids are kept open or the eyes held fixed for even a short period of time.

Persons who need to administer medicine to themselves in the eye may be challenged by anxiety or by a lack of motor skills such as may occur from arthritis or Parkinson's disease. Disability due to age may be another source of difficulty. Small children, for example, may have to be restrained or elderly adults may need assistance from a caregiver to accomplish the goal of getting medication into their eyes.

Medications and irrigations are frequently required for individuals with glaucoma, infections such as conjunctivitis (red-eye), injuries to the eyes or contact lens wearers. In addition, immuno-compromised patients are at risk for requiring eye medication and treatment.

The eyedropper is familiar to everyone as a common apparatus for administering medications to the eye. Eyedroppers apply the medicine as drops on the anterior surface of an eye. Using an eyedropper requires a steady hand and a steady head. For best results with an eyedropper, the patient's head should be tilted back so that the drops fall down onto the eye. The process is often stressful and messy. This is particularly true for the elderly and the very young, precisely those who most commonly need such medications.

There are numerous drawbacks associated with dispensing eye medicine as a drop. The size and the number of drops is difficult to control. It is also difficult to evenly distribute the medicine over the entire anterior surface of the eye. Uniform dosing of the medication, therefore, is rarely achieved with eye drops. Another drawback of eyedrops is anxiety. Anticipation of the drop falling onto the eyeball makes many patients anxious, causing the hand holding the eyedropper to shake or causing the eye to blink. Often, the result is that the drop misses its target. A further drawback of eye drops may be physical discomfort, including pain or irritation, from the sudden increase in liquid volume on the surface of the eye.

Conventional eyedroppers make it difficult to control the quantity of medicine administered to the eye. Excess medicine often spills over the eyelid which is messy and costly. Failure to administer the required dose of medicine to the eye may result in incomplete treatment and may extend the patient's illness.

Typical droppers dispense a drop or two of medicine or fluid onto the eye, or into the cul-de-sac of the eye. Of the one or two drops which are applied, most of the liquid never gets into the eye or the medicine fails to reach the affected tissue. The fluid may run off as tears, may be lost through the tear ducts or it may run down the canillicular track into the throat. Medicine that is lost or that remains in the cul-de-sac where it cannot be distributed over the eye's surface by blinking, becomes unavailable for therapeutic purposes.

Further, the necessity to apply more liquid medication to compensate for the “run-off’ means that medication is wasted. Proper eye-drop technique must be followed or the liquid medication will not cover the eye completely or reach the affected tissue.

Another drawback of standard devices is an inability to control the proper placement of the dropper opening over the eyeball. Improper location of the dropper, nervous jitters, unexpected movement or jarring, may cause accidental poking of the eye with the device and consequent injury to the eye. Young children, the elderly, and the disabled present an especially difficult situation when trying to administer eye medication.

In cases where the eye or eyes are infected, the eyedropper may become contaminated from inadvertent contact with infected tissue or household germs and pass the infection back into the eye or spread the infection to previously uninfected tissues. In families where the eye-medication is shared between siblings, for example, infections such as pink eye may be transmitted through the eyedropper.

Another consideration is non-compliance. Eyedroppers require self-motivated action and coordination from the patient. The mess, anxiety and discomfort of applying drops onto the eyeball frequently leads to non-compliance by the patient with consequent cessation of treatment or only intermittent treatment.

Means for administering medicine to the eye other than an eyedropper are a jet or spray. Jets or sprays fire a rapid burst of medication into the eye before the eye can respond with a recoil or blink. Jets or sprays avoid some of the problems inherent with the eyedropper, but jets are still unpleasant and may cause anxiety when the patient learns that the medication is a surprise delivery.

There remains a long felt, significant and unfulfilled need, therefore, for an ocular treatment apparatus and methods by which medication is passively delivered to the eye so that administration does not require a high degree of manual dexterity to position and operate the apparatus. Additionally, the apparatus should deliver the medication in a manner that does not engender anxiety or physical discomfort. The apparatus should not have projecting members that might physically contact eye tissue. A patient with a weak or unsteady hand should be able to operate the apparatus with ease, and without the potential for injury or infection. There is also a need for an ocular treatment apparatus that is capable of dispensing medicine in the eye in a prescribed, effective, accurate and measured amount. Methods for delivering medication to one or more eyes so that the eyes do not react adversely are also needed.

### SUMMARY OF THE INVENTION

The present invention was designed to apply medication into the eyes of the non-compliant patient such as a small child. It is designed so the user can watch television or read a book while medication is taking place. Liquid medication is atomized or converted by the present invention from a liquid into an aerosol. The aerosol droplet size and velocity produced by the present invention results in a momentum transfer of the medication to the eye tissue, bringing the aerosol droplets into contact with the tissue.

When an eyedropper releases a drop, the mass and the velocity of the drop create momentum (momentum is mass multiplied by velocity). Upon contact with the eyeball, the momentum of the drop creates pressure against the pressure sensors of the eye causing a blink response. The natural tendency of the body is to recoil against this intrusion of the eye. Consequently, most people find the eye drop method difficult and uncomfortable. Significantly, the atomized droplets produced by the present invention impact the eye tissue with a pressure that is below the threshold of sensation of the pressure sensing cells of the eye.

The aerosol produced by the present invention is directed towards the eye or other afflicted tissue. The apparatus maintains a very low momentum of medication through particle size reduction and velocity decrease. At one extreme, however, the invention contemplates an eye or tissue wash or bath where there is no atomization of the medication liquid so that the tissue is immersed in liquid.

There are a variety of ways to convert liquid to an aerosol (i.e., atomize), including, nebulization, ultrasound, high Speed stirring, conventional spray technology and airless spray technology.

There are also a variety of ways to reduce particle velocity, including adapting the fluid conduit size. Increasing the conduit size decreases the particle velocity. Reducing the pressure of the fluid stream is another way to reduce particle velocity.

The aerosol may be directed to an area on the targeted tissue by shrouding or enveloping the medication stream with a high volume of low pressure air. Another way to direct the aerosol may be with the use of a passive or active turbine at the or near the exit of the atomizer to create a tornado effect with the aerosol particles.

Another method is the fogged mask method, wherein a mask around the eyes is filled with an aerosol fog produced, for example, by a low pressure aerosol nebulizer. The eyes receive moisture carrying medication from the high concentration of aerosol particles contained by the mask.

The present invention provides an apparatus for delivering medicine to one or more tissues. The apparatus includes a mask adapted to be worn around one or more tissues. The mask has and one or more fog outlets proximate to one or more tissues. The mask may further include a substantially transparent mask face. One or more atomizers to atomize medicine into a medicine-carrying fog are in fluid communication with the fog outlets such that the fog discharges from the fog outlets to deliver medicine carried by the fog to one or more tissues. Additionally, the apparatus may include a power supply to supply power to the atomizer, a propellant source in fluid communication with the atomizer and one or more conduits to conduct the fog from the atomizer to the mask fog outlets.

The present invention also provides methods for delivering medicine to the eyes. The methods involve nebulizing one or more medications to form a medication fog and contacting the medication fog with one or more eyes at low fog pressure and high fog volume. The method may further include containing the medication fog near one or more eyes and viewing an image during medication administration.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 is a perspective view of one embodiment of an apparatus of the present invention.

FIG. 2 is a perspective view of an alternative embodiment of the apparatus of FIG. 1.

FIG. 3 is a perspective view of an alternative embodiment of the apparatus of FIG. 2.

FIG. 4 is a perspective view of an alternative embodiment of the apparatus of FIG. 4.

FIG. 5 is a perspective view of another embodiment of an apparatus of the present invention.

FIG. 6 is a perspective view of an alternative embodiment of the apparatus of FIG. 5.

FIGS. 7A-D is rear views of various embodiments of a mask of the apparatus of the present invention.

FIGS. 8A-B is front views of two embodiments of fluid conduits of an apparatus of the present invention.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

A fog is defined, for purposes of the present disclosure, as a liquid suspended in a vehicle, such as air, that supports the liquid fog droplets. To be suspended by air, fog droplets are typically in the size range of microns in diameter. A suitable size range of fog droplets for the present invention is 3 to 5 microns in diameter. In alternative embodiments, the size range of droplets may increase or decrease. In the same volume of air, if the size of the particles is increased, there is a resultant decrease in velocity. The fog droplet size and velocity, together with the relatively large volume of the fog are such that the patient is substantially unaware that the patient's eyes are being medicated because of the low impact momentum of the droplets on the eye tissue. Yet, due to the relatively high density of the medicated fog droplets, an effective amount of medication is delivered to the eyes.

For purposes of the present disclosure, the terms aerosol and fog may be used interchangeably. Likewise, the terms atomize and nebulize may be used interchangeably, as may the terms atomizer and nebulizer. Among the various atomizers that may be adapted for use with the present invention are included perfume aspirators, manual pumps, humidifiers, ultrasonic nebulizers, electrically charged screens or membranes, ultrasonic plates, and pressure canisters of compresses air, nitrogen or carbon dioxide.

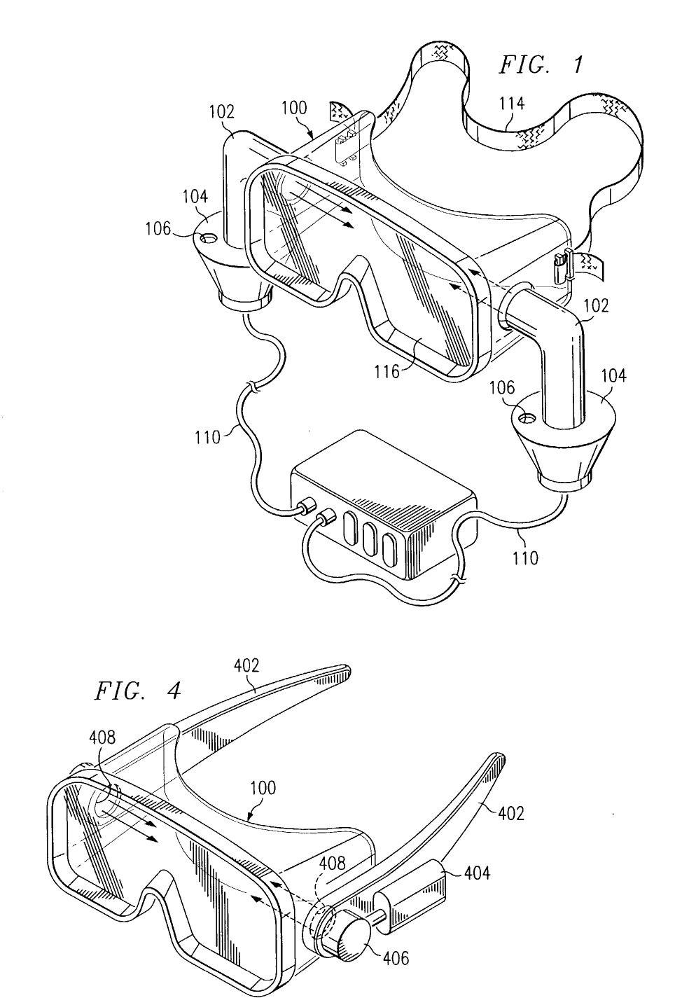

FIG. 1 depicts one embodiment of an apparatus of the present invention. Mask 100 is adapted to fit around and enclose the eyes of a patient. One or more conduits 102 permit fluid flow into the enclosure defined by mask 100. One or more containers 104, in fluid communication with conduits 102, may be adapted to hold fluid such as liquid medication. One or more fill holes 106 may be provided for filling containers 104 with fluid. One or more fluid atomizers or nebulizers 108 may be positioned in or on container 104 such that fluid in container 104 may be atomized to form a liquid particulate fog. One or more electrical leads 110 provide power to atomizer 108 from power supply 112. Power supply 112 may have control functions to modulate various parameters of nebulization such as time, force, frequency and so forth.

Mask 100 may be secured around the eyes with elastic band 114. Mask face 116 is substantially transparent so that the patient may see through it.

In operation, the apparatus of FIG. 1 creates a fog of liquid particles that carry dissolved, suspended or emulsed medication when power 112 is turned on. The medicament fog is impelled, as indicated by the directional arrows in FIG. 1, by nebulizer 108 through conduit 102 into the enclosure defined by mask 100 around the eyes of a patient. The patient may look through mask face 116 to watch television, for example, or to read while the patient's eyes passively take up the medication from the enclosed fog.

The small particle size of the fog droplets and the weak force with which the fog is introduced into the mask enclosure are such that the patient's eyes are virtually unperturbed and have substantially no sensation that the eyes are taking up the medicine. Reading or watching television ensures that the patient's eyes remain open, except for normal blinking, during treatment with the apparatus so that the prescribed dosage of medication is administered to the eyes.

Atomization or nebulization of the liquid may be achieved by any suitable means known in the art. Examples of such means include ultrasound (ultrasonic atomization), jet atomization, nozzles such as a venturi nozzle, sprayers, compressed gas propellant forced through an orifice, injection, vibration such as, for example, membrane or diaphragm vibration, aspiration, bellows, and stirring.

Mask 100 may be fabricated by any of a variety of suitable materials, including plastic, vinyl, composites, light weight metals and the like. Mask 100 may be formed by any of a variety of suitable processes including, for example, injection mold. Mask 100 may be adapted from a pre-formed mask such as a paintball or motocross mask or helmet. In fact, mask 100 may be adapted from a helmet that is worn around the head as well as the face. Alternatively, mask 100 may be a simple frame that holds one or more fog outlets in position to fog an intended medication target such as one or more eyes.

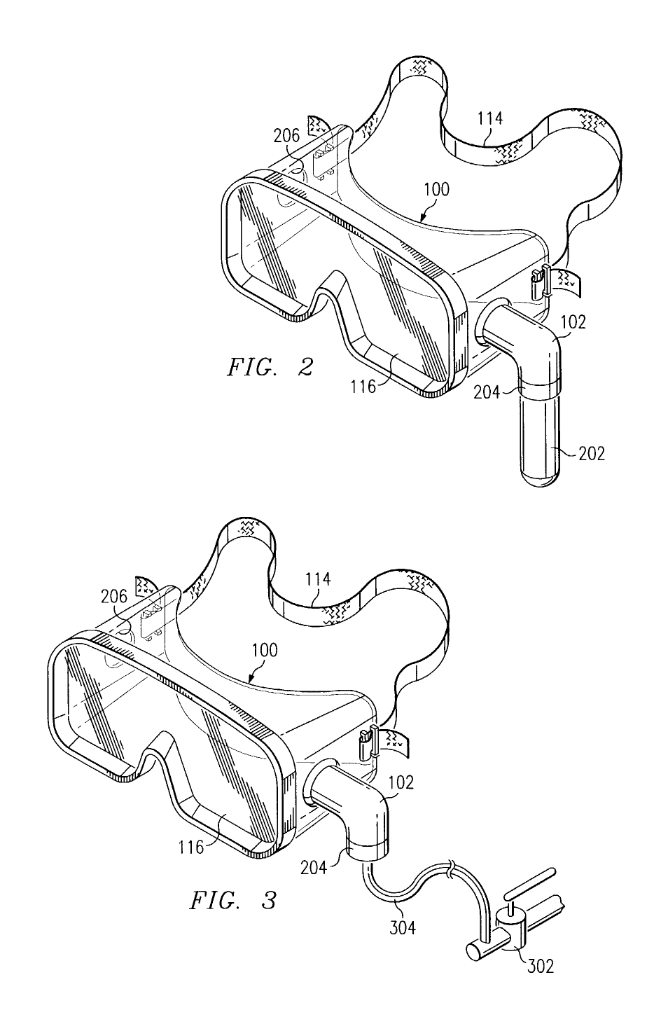

FIG. 2 depicts a variant embodiment of the apparatus of FIG. 1. In the embodiment of FIG. 2, propellant source 202 is fluidly connected to medication chamber 204 which is, in turn, in fluid communication with conduit 102. Medication chamber 204 includes an atomizing orifice or nozzle. Fog outlet orifice 206 distal from conduit 102 may be selectively open or closed.

In operation, medication in medication chamber 204 is atomized by being propelled through an atomizing orifice by propellant source 202 into conduit 102 and the mask enclosure where the eyes are exposed to the medication fog. The medication fog may traverse the mask enclosure and exit orifice 206 if orifice 206 is open. The fog may be contained in the enclosure if orifice 206 is closed. Propellant source 202, medication chamber 204 and conduit 102 may define a fog-generating unit. The unit may be installed on mask 100 optionally at orifice 206 or distal from orifice 206.

FIG. 3 depicts a variant embodiment of the embodiment of FIG. 2. In the embodiment of FIG. 3, propellant source 302 is in fluid communication with medication chamber 204 through propellant conduit 304.

FIG. 4 depicts an embodiment of the apparatus in which the mask is worn like a pair of glasses or spectacles. Instead of bands 114, mask 100 is supported around the eyes by ear support members 402. The apparatus of this embodiment dispenses with conduit 102. Propellant source 404 in fluid communication with medication chamber 406, which includes an atomizing nozzle, propels the medication fog through fog outlet 408 into the mask enclosure.

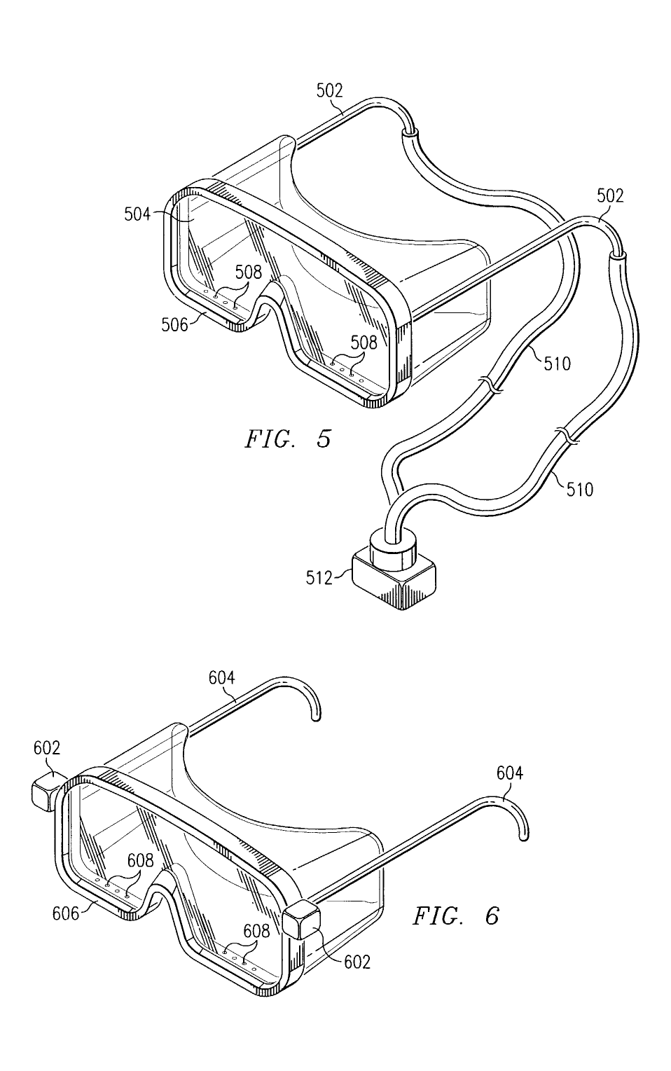

FIG. 5 depicts another embodiment of the invention. In the embodiment of FIG. 5, ear support members 502 may be hollow to provide a fog conduit to mask or mask face 504. Mask face frame 506 may also be hollow such that hollow ear support members 502 are in fluid communication with frame 506. Frame 506 may be perforated intermittently along the surface of frame 506 proximate a patient's eyes to provide fog outlets 508. Flexible Y-shaped conduit 510, in fluid communication with hollow ear support members 504 and nebulizing unit 512, transmits medication-carrying fog from nebulizing unit 512 to ear supports 502.

Nebulizing unit 512 may include one or more medication chambers, one or more liquid atomizers such as an ultrasonic atomizer or a nozzle, and a propellant source such as a pump or a compressed gas canister. Unit 512 may further be operably connected to a power supply such as a battery or an electrical outlet. In operation, nebulizing unit 512 generates a fog of medicine-carrying liquid droplets that pass through conduit 510 to ear supports 502, through ear supports 502 to frame 506 where the fog is discharged out of perforations 508 proximate to a patient's eyes.

FIG. 6 depicts an embodiment of the invention that is a variation of the embodiment of FIG. 5. The embodiment of FIG. 6 represents a substantially portable embodiment where nebulizing units 602, substantially as describe above in FIG. 5, are mounted on ear supports 604 or frame 606, also substantially as described in FIG. 5, at a fog orifice such that a medicinal fog generated by unit 602 passes directly into frame 606 or supports 604 to be discharged from fog outlets 608 spaced along the surface of frame 606 proximate a patient's eyes. For mobile use of the embodiment of FIG. 6, nebulizing unit 602 may include a self-contained power supply such as a battery.

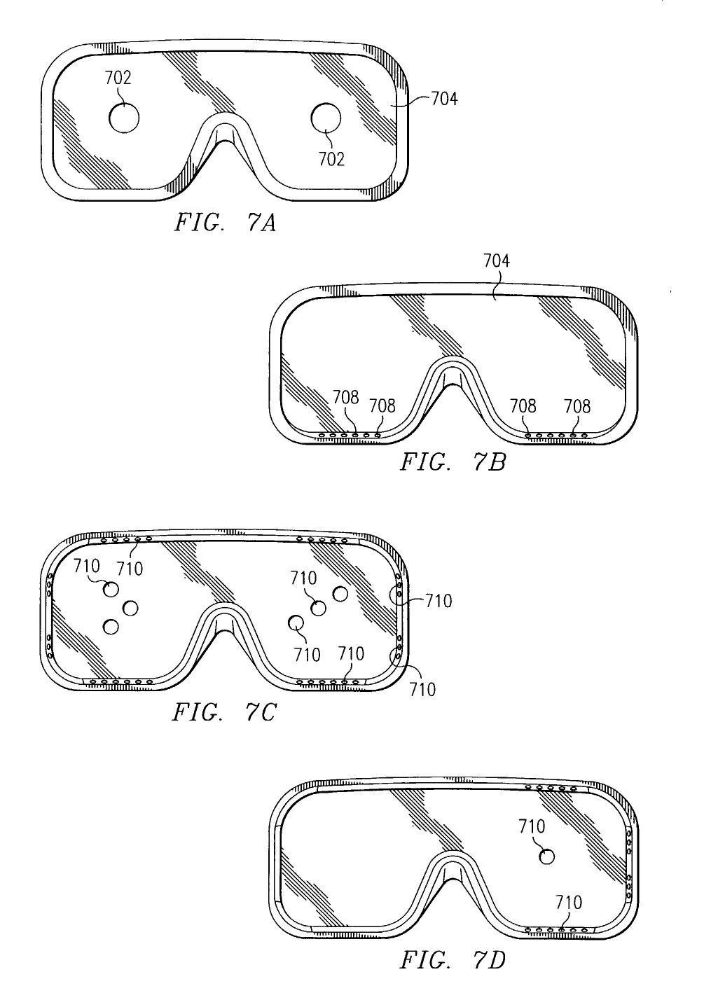

FIGS. 7A-D depicts various embodiments for the placement of fog discharge outlets on the mask or mask face of the present invention on the side proximate to a patient's eyes. In FIG. 7A, fog outlets 702 are placed on mask face 704 bilaterally substantially in front a patient's eyes. A fog conduit mounted on outlets 702 may conduct atomized medicinal fog from a nebulizing unit to outlets 702. In the various embodiments described herein, it will be understood by those skilled in the art that a desired fog discharge pattern may be selected by optionally selecting a desired fog outlet profile such as, for example, circular, ovoid, rectangular and so forth. The present invention contemplates embodiments in which the shape of the fog outlet orifice may be optionally selectable.

In FIG. 7B, mask face 704 is rimmed by frame 706. At least a portion of frame 706 is hollow to provide a fog conduit to one or more fog outlets 708, here placed along the lower edge of frame 706. A nebulizing unit may be connected to the hollow portion of frame 706 by a conduit or by direct mounting such that fluid communication is established from the nebulizer to outlets 708.

FIG. 7C illustrates that one or more fog outlets 710 may be placed at any desired location on the mask face 704 or frame 706 or both. FIG. 7D illustrates that fog outlets 710 need not be placed bilaterally.

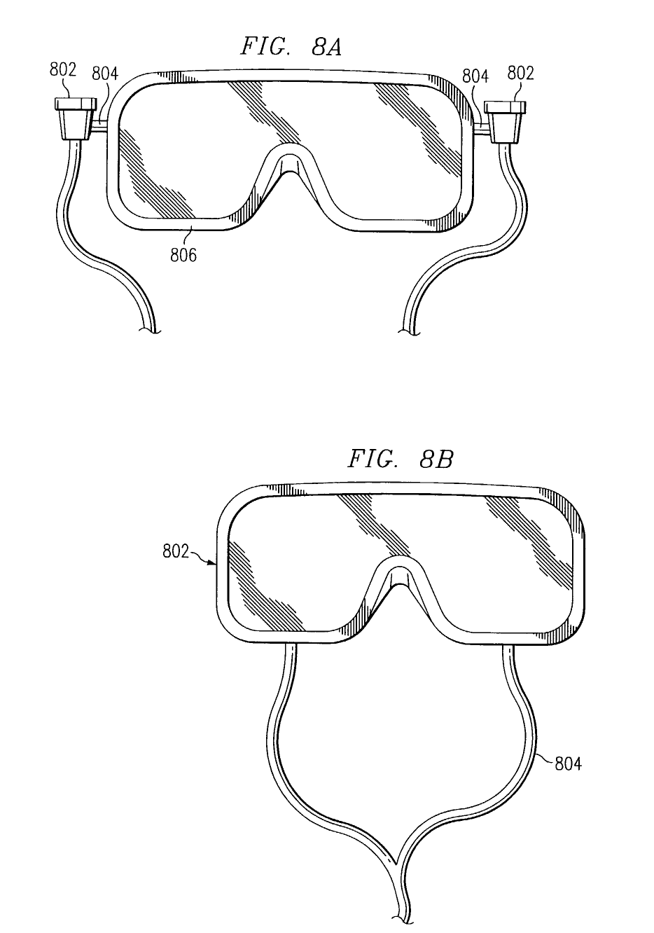

FIGS. 8A and 8B illustrate structures for transmitting an aerosol fog from a nebulizer to the mask of the present invention. In FIG. 8A, nebulizers 802 are mounted with fog conduits 804 to frame 806 around mask face 808. Propellant conduits 810 supply propellant to nebulizing unit 802 which may include a medication chamber for liquid medication and an atomizing nozzle, for example, such that propellant impels the liquid medicine through the atomizing nozzle to create a fog or aerosol that is discharged from fog conduit 804 into the air around the eyes of a patient. Mask face 808 and frame 806 substantially or partially contain the medicinal fog near to the eyes.

In FIG. 8B, a medicinal aerosol fog is transmitted from a nebulizer (not shown) to mask 802 by Y-shaped fog conduit 804 which discharges the fog into the air near a patient's eyes. Fog conduit 804 may be of any suitable shape, including Y-shaped or L-shaped. In the embodiment of FIG. 8B, fog conduit 804 is mounted on the lower edge of mask frame 806, conduit 804 may be mounted on mask 802 at any suitable location provided that the medicinal fog is discharged near the eyes is at least partially contained near they eyes by mask 802.

For purposes of the present disclosure, the term mask is used to refer to any suitable means for confining the fog near one or more eyes. The apparatus of the present invention may confine the medication fog near the eyes with a mask, a diving mask, goggles, swimmers goggles, UV tanning goggles, eyecups, spectacles and so forth. The mask may be adapted to form at least a partial seal around the eyes to enhance the fog confinement. The mask may be further adapted to provide vents so that the fog flows across the eyes.

To view an image through the mask, the mask may have a substantially transparent or translucent mask face or faceplate. Suitable materials for the mask face include acrylic and polycarbonate.

The transparent faceplate allows the patient to view an image during administration of medication. A caregiver is able to inquire whether the patient recognizes a particular image and thereby verify that the patient's eyes were open during treatment. The image may be a photograph or a writing sample. The image may be provided by television or computer screen. Further, the patient may control the image or changes images. Some patients may enjoy playing a video game, for example, during treatment in which game the patient may be so engrossed that the patient is unaware that treatment is taking place. In another embodiment, the patient with a video game joystick or controller, for example, may trigger administration of the medicated fog by an apparatus of the present invention.

The foregoing description has been directed to particular embodiments of the invention in accordance with the Patent Statutes for the purposes of illustration and explanation. Many alternatives, modifications, and variations will be apparent to those skilled in the art in light of the foregoing disclosure. In particular, changes may be made to the shape and size of the apparatus to accommodate all patients, including humans and animals.

What is claimed is:

1. An apparatus for delivering medicine to one or more tissues, the apparatus comprising:
a mask adapted to be worn around one or more tissues, the mask further comprising one or more fog outlets proximate to one or more tissues; and
one or more atomizers to atomize medicine into a medicine-carrying fog, wherein one or more atomizers are in fluid communication with one or more fog outlets such that the fog discharges from one or more fog outlets to deliver medicine carried by the fog to one or more tissues.

2. The apparatus of claim 1, wherein one or more atomizers comprise an ultrasonic atomizer.

3. The apparatus of claim 1, wherein one or more atomizers comprise a nozzle.

4. The apparatus of claim 1, wherein one or more atomizers comprise a nebulizer.

5. The apparatus of claim 1, wherein one or more atomizers comprise a stirrer.

6. The apparatus of claim 1, wherein one or more atomizers further comprise one or more medication chambers.

7. The apparatus of claim 1, further comprising one or more conduits in fluid communication with one or more atomizers and one or more fog outlets.

8. The apparatus of claim 1, wherein the fog comprises liquid droplets in the size range of approximately 3 to 5 microns in diameter.

9. The apparatus of claim 1, further comprising one or more propellant sources in fluid communication with one or more atomizers to propel the fog to one or more fog outlets.

10. The apparatus of claim 9, wherein one or more propellant sources comprise a compressed gas canister and the gas compressed in one or more of the canisters is selected from the group consisting of air, nitrogen or carbon dioxide.

11. The apparatus of claim 9, wherein one or more propellant sources comprise a pump.

12. The apparatus of claim 1, further comprising one or more power supplies connected to one or more atomizers.

13. The apparatus of claim 12, wherein one or more power supplies comprise one or more batteries.

14. The apparatus of claim 12, wherein one or more power supplies comprise one or more electric wall outlets.

15. An apparatus for delivering medicine to one or more eyes, the apparatus comprising:
a mask adapted to be worn around one or more eyes, the mask further comprising a substantially transparent mask face and one or more fog outlets proximate to one or more eyes;
one or more atomizers to atomize medicine into a medicine-carrying fog, one or more atomizers further comprising one or more medication chambers to contain one or more medicines;
a power supply to supply power to the atomizer;
a propellant source in fluid communication with the atomizer and one or more fog outlets; and
one or more conduits in fluid communication with the atomizer and with one or more fog outlets such that the fog may pass from the atomizer through one or more conduits to the mask and be discharged from one or more fog outlets to deliver medicine carried by the fog to at least one eye.

16. The apparatus of claim 15, wherein one or more conduits comprise one or more flexible conduits.

17. The apparatus of claim 15, wherein the power supply comprises a battery.

18. The apparatus of claim 15, wherein the fog comprises liquid droplets in the size range of approximately 3 to 5 microns in diameter.

19. A method for administering one or more medications to one or more tissues, the method comprising:
nebulizing one or more medications to form a medication fog; and
contacting the medication fog with one or more tissues at low fog pressure and high fog volume.

20. The method of claim 19, wherein one or more of the tissues comprises one or more eyes, the method further comprising:
containing the medication fog near one or more eyes and
viewing an image with one or more eyes during medication administration.
